(19) United States
(12) Patent Application Publication
(10) Pub. No.: US 2024/0055135 A1
(43) Pub. Date: Feb. 15, 2024

(54) SYSTEM AND METHOD FOR MEASURING AN INDIVIDUAL'S HEALTH AND FITNESS LEVELS

(71) Applicant: Fiziolog, LLC, Pleasant Ridge, MI (US)

(72) Inventor: Austin Evans, Royal Oak, MI (US)

(73) Assignee: Fiziolog, LLC, Pleasant Ridge, MI (US)

(21) Appl. No.: 18/270,748

(22) PCT Filed: Apr. 1, 2022
(86) PCT No.: PCT/US2022/011107

§ 371 Date: Jul. 3, 2023

Related U.S. Application Data
(60) Provisional application No. 63/133,868, filed on Jan. 5, 2021.

(51) Int. Cl. G16H 50/30; G16H 10/60; G16H 50/70

(52) U.S. Cl. CPC G16H 50/30; G16H 10/60; G16H 50/70

(57) ABSTRACT

A system and methods for quantifiably measuring an individual's health and fitness levels for a plurality of health and fitness categories and comparing the measured levels to an individual's demographic group (e.g. age and gender) based on currently available and reliable scientific data and information. The result is an overall picture of an individual's health and fitness levels.

### CROSS REFERENCE TO RELATED APPLICATION

The present application claims the benefit to United States Provisional Patent Application No. 63/133,868 filed on Jan. 5, 2021, which is incorporated herein by reference in its entirety.

### FIELD

The present disclosure relates to a system and method for quantifiably measuring an individual's health and fitness levels.

### BACKGROUND

People are now more interested than ever in monitoring, tracking, assessing, and improving their health and fitness levels. In order to accomplish this, it is important for them to obtain an accurate representation of where they currently stand among their respective demographic groups. People can then use this information to easily identify the specific areas in which they can improve their health and fitness levels.

Currently available systems and methods provide individuals with data and information pertaining to only one or two components of health and fitness attributes. For example, some products only provide individuals with data analysis on components related to cardiovascular values and activity tracking, such as heart rate, distance, calories, steps, etc. However, these systems and methods do not provide individuals with a larger (i.e. macro) view of their particular overall health and fitness levels. Also, current systems and methods fail to quantifiably assess/score a plurality of categories of health and fitness for the individual based on currently available and reliable comparative data.

Consequently, there is a need for a system and method that can provide individuals with the ability to visualize a complete picture of their overall health and fitness abilities in a scientifically quantifiable way.

### SUMMARY

What is provided is a system and methods for quantifiably measuring an individual's health and fitness levels for a plurality of health and fitness categories and comparing the measured levels to an individual's demographic group (e.g. age and gender) based on currently available and reliable scientific data and information. The result is an overall picture of an individual's health and fitness levels.

In an embodiment, the system comprises a first computing device in communication with a second computing device via a network. The first computing device includes a display, one or more input/output devices, one or more processors, and memory. The second computing device comprises one or more processors/microprocessors capable of performing tasks, such as all or a portion of the methods described herein. The second computing device further includes an engine having a user account module configured to build user profiles and to authenticate users in the system; a data analysis module in communication with the user account module, wherein the data analysis module is configured to quantifiably analyze a first set of data and information based on a plurality of health and fitness categories; a comparative analysis module in communication with the data analysis module, wherein the comparative analysis module is configured to review and evaluate a second set of data and information from one or more external sources, and to compare and contrast the analyzed first set of data and information with the second set of data and information; and a score computing module in communication with the comparative analysis module, wherein the score computing module is configured to generate a score associated with each of the health and fitness categories based on the input received from the comparative analysis module.

In an embodiment, a method for measuring an individual's health and fitness levels includes creating a new session to receive a first set of data and information pertaining to an individual's health and fitness; analyzing the first set of data and information to generate averages and to compute scores based on a plurality of health and fitness categories; identifying and screening a second set of data and information from one or more external sources; comparing and contrasting the analyzed first set of data and information with the second set of data and information; and generating a total score for the individual based on computed scores for each of the plurality of health and fitness categories.

In some embodiments, the system uses five main categories for assessing the health and fitness levels of a user: (1) body composition; (2) balance; (3) cardiovascular/cardiopulmonary fitness; (4) muscular fitness; and (5) range of motion. Each of these categories may include a plurality of components that affect the overall assessment for each of the categories. The total average for each category may be computed based on quantified and weighted averages of the various components associated with each category.

In some embodiments, the second set of data and information (i.e., comparative data) is translated into a demographic-specific percentile range.

### BRIEF DESCRIPTION OF THE DRAWINGS

The above, as well as other advantages of the present disclosure, will become readily apparent to those skilled in the art from the following detailed description when considered in light of the accompanying drawings in which:

FIG. 1 is a diagram illustrating an example system for measuring an individual's health and fitness levels according to an embodiment of the present disclosure;

FIG. 2 is a block diagram illustrating example modules of a health and fitness analysis engine illustrated in FIG. 1;

FIG. 3 is a flow chart illustrating an example method for measuring an individual's health and fitness levels using the system illustrated in FIG. 1;

FIG. 4 is a chart illustrating an example of the usage of the system illustrated in FIG. 1;

FIG. 5 is a graphical user interface (GUI) illustrating an example summary page of data quantified by the system illustrated in FIG. 1 using a plurality of categories;

FIG. 6 is a GUI illustrating an example body composition summary page for a user generated by the system illustrated in FIG. 1;

FIG. 7 is a GUI illustrating an example balance summary page for a user generated by the system illustrated in FIG. 1;

FIG. 8 is a GUI illustrating an example cardiovascular/cardiopulmonary fitness summary page for a user generated by the system illustrated in FIG. 1;

FIG. 9 is a GUI illustrating an example muscular fitness summary page for a user generated by the system illustrated in FIG. 1;

FIG. 10 is a GUI illustrating an example range of motion summary page for a user generated by the system illustrated in FIG. 1;

FIG. 11 is a GUI illustrating an example data input summary page for each of the categories generated by the system illustrated in FIG. 1;

FIG. 12 is a GUI illustrating an example body composition input page generated by the system illustrated in FIG. 1;

FIG. 13 is a GUI illustrating an example balance input page generated by the system illustrated in FIG. 1;

FIG. 14 is a GUI illustrating an example cardiovascular/cardiopulmonary fitness input page generated by the system illustrated in FIG. 1;

FIG. 15 is a GUI illustrating an example muscular fitness input page generated by the system illustrated in FIG. 1;

FIG. 16 is a GUI illustrating an example range of motion input page generated by the system illustrated in FIG. 1; and

FIG. 17 is a GUI illustrating an example comparison page by a category and a component of the category, including weighted average examples and reference material.

### DETAILED DESCRIPTION

It is to be understood that the disclosure may assume various alternative orientations and step sequences, except where expressly specified to the contrary. It is also understood that the specific devices and processes illustrated in the attached drawings, and described in the specification are simply exemplary embodiments of the inventive concepts disclosed and defined herein. Hence, specific dimensions, directions or other physical characteristics relating to the various embodiments disclosed are not to be considered as limiting, unless expressly stated otherwise.

Some portions of the detailed description that follow are presented in terms of algorithms and/or symbolic representations of operations on data bits and/or binary digital signals stored within a computing system, such as within a computer and/or computing system memory. An algorithm is here and generally considered to be a self-consistent sequence of operations and/or similar processing leading to a desired result. The operations and/or processing may take the form of electrical and/or magnetic signals configured to be stored, transferred, combined, compared and/or otherwise manipulated. It has proven convenient at times, principally for reasons of common usage, to refer to these signals as bits, data, values, elements, symbols, characters, terms, numbers, numerals and/or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing”, “computing”, “calculating”, “determining” and/or the like refer to the actions and/or processes of a computing platform, such as a computer or a similar electronic computing device that manipulates and/or transforms data represented as physical electronic and/or magnetic quantities and/or other physical quantities within the computing platform's processors, memories, registers, and/or other information storage, transmission, and/or display devices.

Unless specifically stated otherwise, as apparent from the following discussion, it is appreciated that throughout this specification a computing platform/computing system includes, but is not limited to, a device such as a computer or a similar electronic computing device that manipulates and/or transforms data represented by physical, electronic, and/or magnetic quantities and/or other physical quantities within the computing platform's processors, memories, registers, and/or other information storage, transmission, reception and/or display devices. Accordingly, a computing platform refers to a system, a device, and/or a logical construct that includes the ability to process and/or store data in the form of signals. Thus, a computing platform, in this context, may comprise hardware, software, firmware and/or any combinations thereof. A user may include an end-user.

Flow charts, also referred to as flow diagrams by some, are used in some figures herein to illustrate certain aspects of some examples. Logic they illustrate is not intended to be exhaustive of any, all, or even most possibilities. Their purpose is to help facilitate an understanding of this disclosure with regard to the particular matters disclosed herein. To this end, many well-known techniques and design choices are not repeated herein so as not to obscure the teachings of this disclosure. Any number of additional operations may be interposed between or in addition to those disclosed. Furthermore, the order of operations presented is not intended to be rigid or exclusive.

Throughout this specification, the term “system” may, depending at least in part upon the particular context, be understood to include any method, process, apparatus, and/or other patentable subject matter that implements the subject matter disclosed herein. The subject matter described herein may be implemented in software, in combination with hardware and/or firmware. For example, the subject matter described herein may be implemented in software executed by a hardware processor.

The aspects and functionalities described herein may operate via a multitude of computing systems, wired and wireless computing systems, mobile computing systems (e.g., mobile phones, tablets, watches, notebooks, and laptop computers), desktop computers, hand-held devices, multiprocessor systems, and the like.

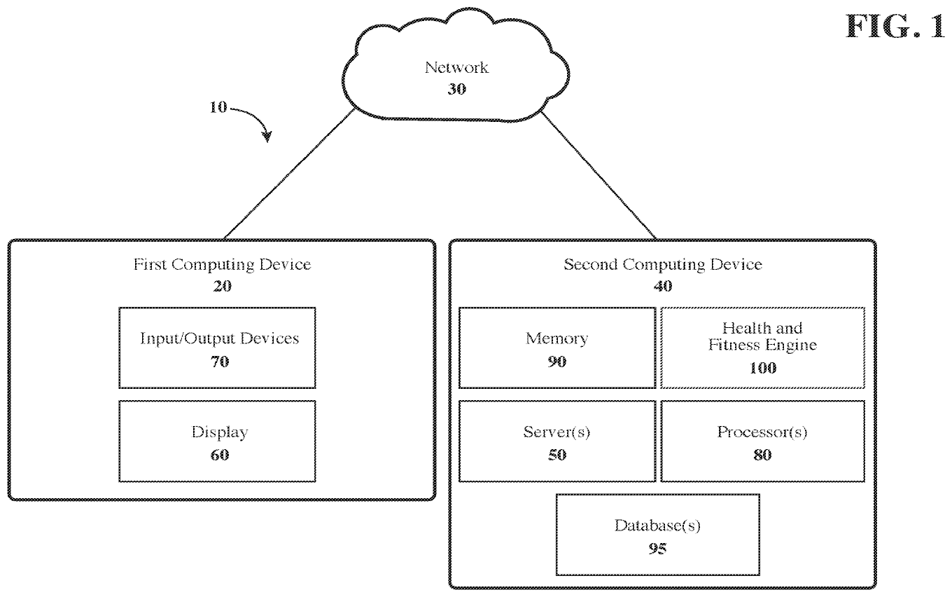

FIG. 1 illustrates a block diagram showing an example system 10 for measuring an individual's health and fitness levels according to an embodiment of the present disclosure. The system 10 comprises a first computing device 20, a communications network 30, and a second computing device 40. The first computing device 20 may communicate with the second computing device 40 using the network 30, such as a wireless “cloud network,” the Internet, an IP network, or the like. In an alternative embodiment, the first computing device 20 may be connected to the second computing device 40 using a hard-wired connection.

The first computing device 20 may be a laptop, tablet, cellular phone, handheld device, watch, or any other functionally equivalent device capable of accessing the Internet using a browser. In an embodiment, the first computing device 20 includes, a display 60, one or more input/output devices 70, one or more processors, and memory. The display 60 may be a visual display, such as a screen, that is built-in to the first computing device 20. The input/output devices 70 may be a keyboard, a mouse, a pen, a touch input device, etc.

The second computing device 40 comprises one or more servers 50, such as web servers, database servers, and application program interface (API) servers. The second computing device 40 may be a desktop computer, a laptop, a tablet, a server computer, or any other functionally equivalent device known in the art. The second computing device 40 also comprise one or more processors/microprocessors 80 capable of performing tasks, such as all or a portion of the methods described herein. The second computing device 40 further comprises memory 90. The memory 90 includes computer-readable instructions that may include computer-readable storage media and computer-readable communication media. The memory 90 may be any type of local, remote, auxiliary, flash, cloud, or other memory known in the art.

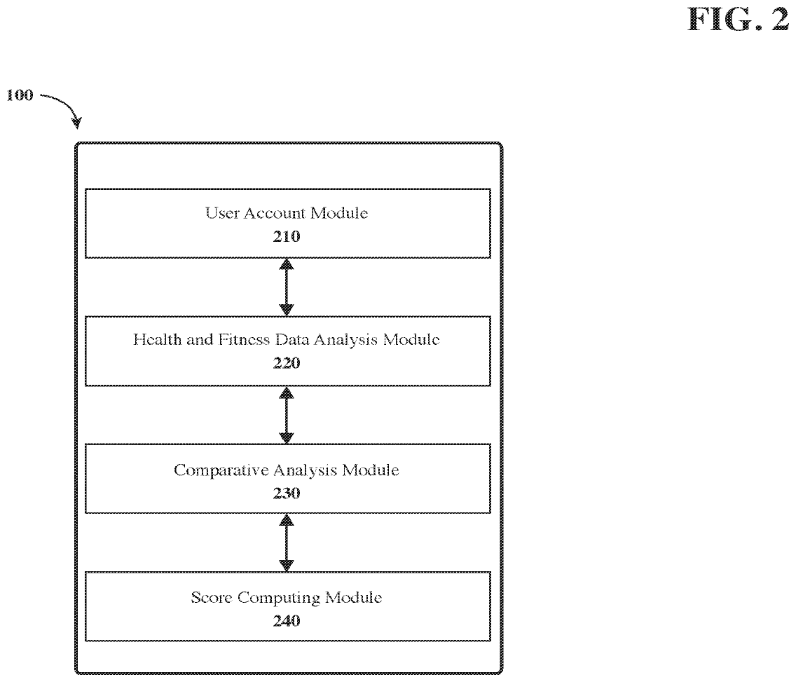

In some embodiments, the memory 90 comprises, but is not limited to, random access memory, read-only memory, flash memory, or any combination of such memories. In some embodiments, the memory 90 includes one or more program modules suitable for running software modules, such as a health and fitness engine 100, as shown in FIGS. 1 and 2. The health and fitness engine 100 includes one or more processors to perform the methods described herein.

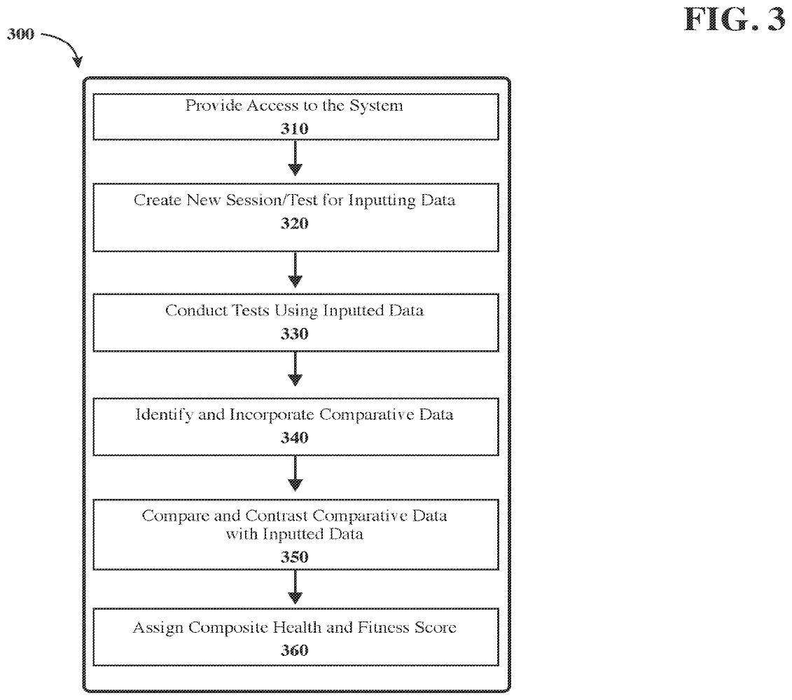

A number of program modules and data files are stored in the memory 90, The memory 90 provides non-volatile, non-transitory storage for the second computing device 40. While executing on the processors 80, program modules depicted in FIG. 2 perform processes including, but not limited to, one or more of the steps of the method 300 illustrated in FIG. 3, as described below.

The second computing device 40 further comprises one or more databases 95 associated with the one or more servers 50. The databases 95 are configured to store all data related to the system 10, including, but not limited to, health and fitness data and information inputted into the system 10.

FIG. 2 illustrates a block diagram showing example modules of the health and fitness engine 100 illustrated in FIG. 1. The health and fitness engine 100 is configured to analyze and quantify data related to one or more health and fitness categories of an individual; to compare the individual's health and fitness to currently-updated and reliable data for an individual's demographic group; and to aggregate the compared data to generate averages and scores for the individual. Examples of the modules include a user account module 210, a health and fitness data analysis module 220, a comparative analysis module 230, a score computing module 240, and combinations thereof.

The user account module 210 is configured to receive information from users of the system and to build user profiles and authenticate users based on the user information. In an embodiment, the system 10 is used by an individual user administrator to input personal data and information, such as their date of birth, their gender, and information about themselves related to a plurality of health and fitness categories and components of such categories. The individual user administrator may select the amount of information they want to test and calculate using the system 10. The individual user administrator may also view and edit reports regarding the analysis of their health and fitness levels, including being able to compare themselves to their demographic group.

In another embodiment, the system 10 is used by a group user administrator that is affiliated with one or more individual users. For example, the group user administrator may be a gym or similar organization, while the individual users within the group may be members of the gym/organization. In this example, the individual users may access data and view reports, but only the group user administrator may edit the accessed/inputted data and reports.

The health and fitness data analysis module 220 is in communication with the user account module 210 and is configured to analyze the data and information provided by users of the system 10. Specifically, the health and fitness data analysis module 220 quantifiably analyzes the data and information provided by the user based on various health and fitness categories/metrics, calculations, and algorithms adopted by the system 10.

In an embodiment, the health and fitness categories for each user include body composition, balance, cardiovascular/cardiopulmonary fitness, muscular fitness, and range of motion. Each of these health and fitness categories comprise a plurality of components that are adopted by the system 10. The number and type of health and fitness categories may vary in other embodiments.

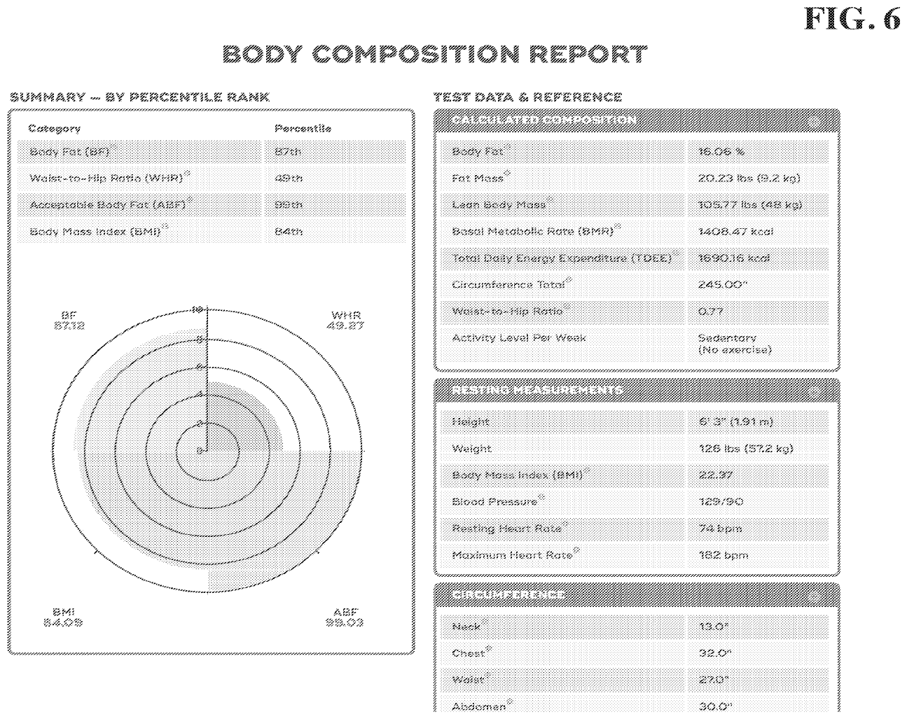

As best seen in FIG. 6, non-limiting examples of components measured in the body composition category include a user's height, weight, body mass index (BMI), body fat, fat mass, lean body mass, basal metabolic rate (BMR), total daily energy expenditure (TDEE), activity level per week, waist-to-hip ratio heart rate, blood pressure, circumferences of one or more body parts of a user one or more skinfold values of a user, and any combinations thereof. One or more of the components of the body composition category may be quantifiably measured and compared with other components of the body composition category by the health and fitness data analysis module 220.

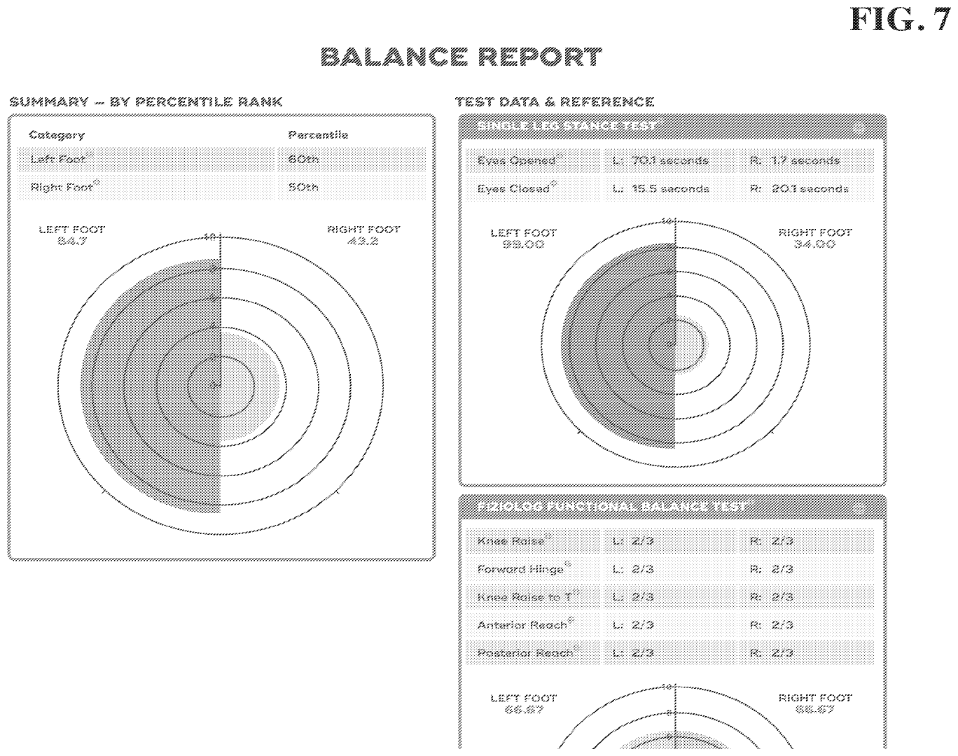

As best seen in FIG. 7, non-limiting examples of components measured in the balance category include tests identifying the abilities of balancing on one leg for a length of time with eyes opened and eyes closed; tests identifying faults or abilities associated with single leg stance, double leg stance, and tandem stance for a user; tests identifying faults or abilities for the left and right feet of a user, based on different movements of the feet and for different portions of a user's feet, such as anterior, posteromedial, and posterolateral portions. One or more of the components of the balance category may be quantifiably measured and compared with other components of the balance category by the health and fitness data analysis module 220.

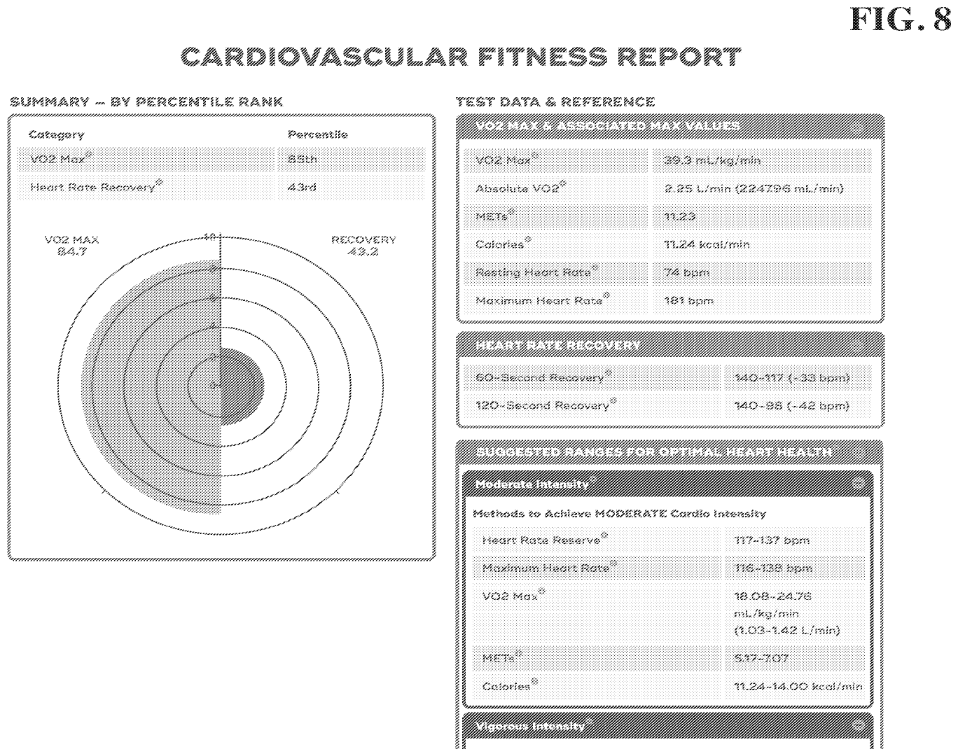

As best seen in FIG. 8, non-limiting examples of components measured in the cardiovascular/cardiopulmonary fitness category include a user's maximum rate of oxygen consumption (VO2 max), absolute rate of oxygen consumption, metabolic equivalents (METs), heart rate recovery, heart rate (resting and maximum), calories, various cardiovascular exercise intensities, and heart rate zones in response to various levels of physical activity. For example, heart rate zones may be established with estimated heart rate ranges for different types of activities. One or more of the components of the cardiovascular/cardiopulmonary fitness category may be quantifiably measured and compared with other components of the cardiovascular/cardiopulmonary fitness category by the health and fitness data analysis module 220.

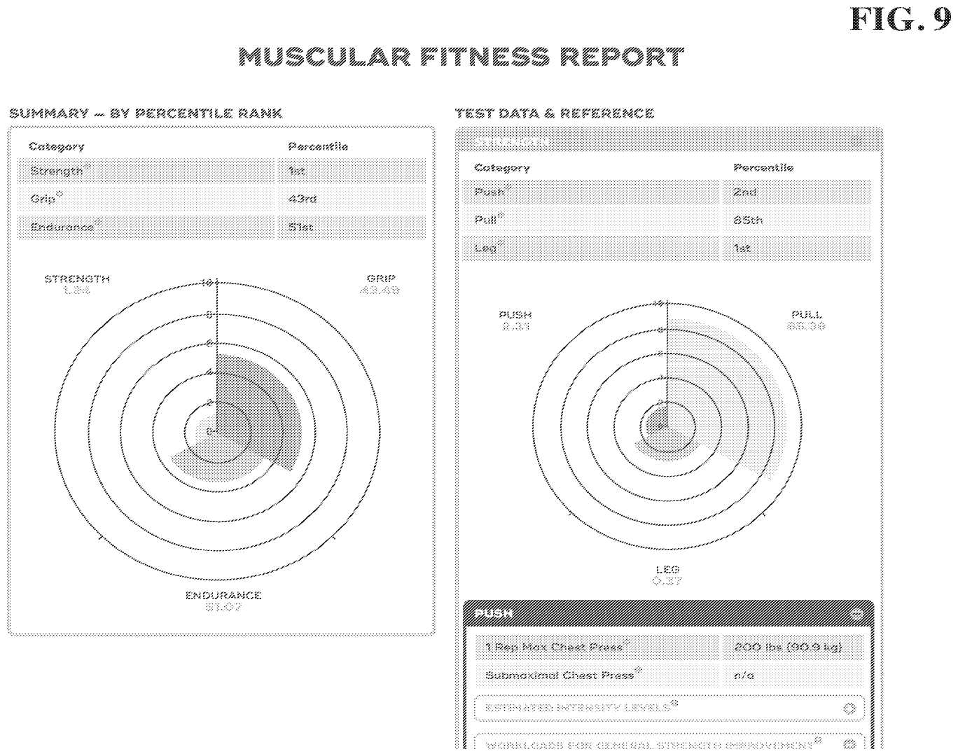

As best seen in FIG. 9, non-limiting examples of components measured in the muscular fitness category include a user's muscular strength (e.g., push, legs, pull), including 1-repetition max (1RM) values, submaximal values, and various ranges of intensity levels, workloads, and repetitions; grip strength for left and right hands; and muscular endurance (e.g., push, pull, leg, core). One or more of the components of the muscular fitness category may be quantifiably measured and compared with other components of the muscular fitness category by the health and fitness data analysis module 220.

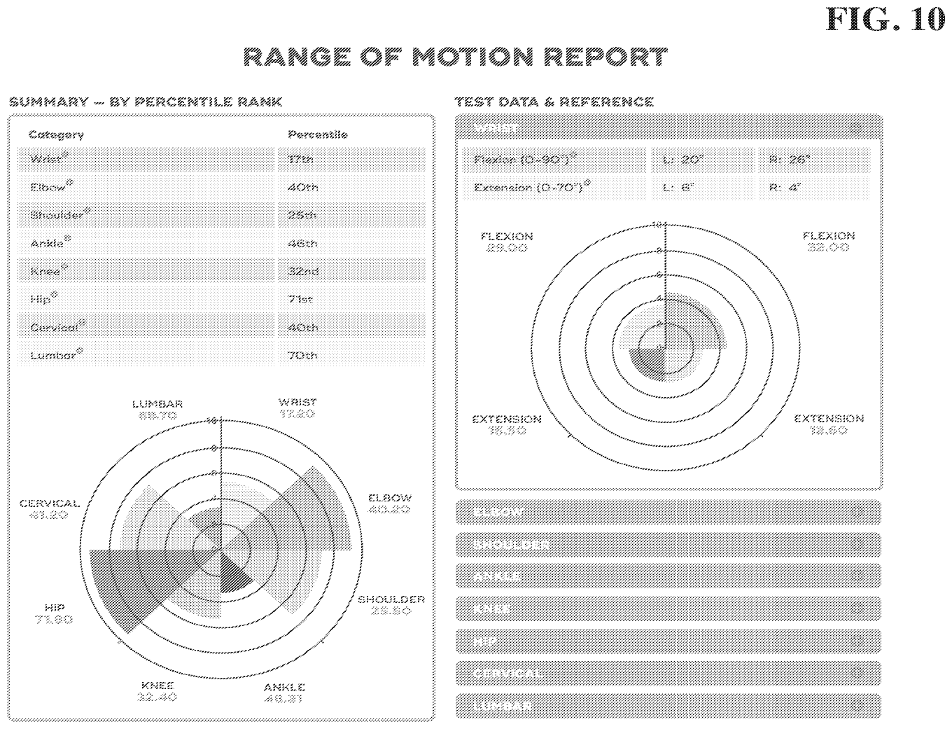

As best seen in FIG. 10, non-limiting examples of components measured in the range of motion category include a range of motion for a user's wrist (flexion and extension), elbow (flexion, extension, pronation, and supination), cervical (flexion, extension, and lateral rotation), shoulder (flexion, extension, abduction, and lateral rotation), lumbar (flexion and extension), hip (flexion, hyperextension, and abduction), knee (flexion and extension), and ankle (dorsiflexion and plantar flexion). One or more of the components of the range of motion category may be quantifiably measured and compared with other components of the range of motion category by the health and fitness data analysis module 220.

The comparative analysis module 230 is in communication with the data analysis module 220 and is configured to filter, select, input, and reference publicly available, reliable, and/or peer-reviewed data (“comparative data”). The comparative data is then translated into a demographic-specific percentile range, such as a 99-1% formula. The comparative data can then be assigned a specific percentile rank. The comparative data is derived from one or more organizations, publications, journals, or academic literature. In an embodiment, portions from a plurality of references may be taken into consideration and averaged to determine the most accurate estimated value for a particular component of a category. The comparative data reflects one or more of the components of the health and fitness categories determined and set by the system 10.

In some embodiments, the system 10 mathematically calculates a specific percentage between 99 and 1 when the comparative data is not already broken down into a 99-1% range. For example, the comparative data may be broken down into categories, such as “Excellent,” “Good,” “Average,” and “Poor,” wherein each expresses a value range. In this example, the “Excellent” category may have a body fat range between 8.5% and 14.2% and the “Poor” category may have a body fat range between 26%-35%. In an embodiment, the system 10 identifies the mathematical integers and is able to accurately assign an actual percentile to each of the values. By way of example, if a particular piece of comparative data identifies the top 90^{th} percentile and the bottom 5^{th} percentile, the system 10 can compute the remaining percentages based on the provided data values.

The comparative analysis module 230 is also configured to cross-reference and compare the data quantified by the health and fitness data analysis module 220 with the comparative data. In an embodiment, the comparative data is associated with specific demographic groups, such as age or gender. As a result, a more accurate representation of a user's health and fitness levels as compared with their demographic group is provided. The goal being to provide the user with a better understanding of where they stand (from a health and fitness perspective) in relation to their demographic group. In order to ensure that the comparative data is current and reliable, the comparative analysis module 230 is configured to consistently and continuously update this data, either automatically or manually, in the databases 95 of the system 10 as it becomes available. As such, the data may be updated in real-time or near real-time.

The score computing module 240 is configured to quantifiably classify and score the health and fitness categories of a user by computing averages for the components associated with each category. In an embodiment, the averages for one or more of the body composition, balance, cardiovascular/cardiopulmonary fitness, muscular fitness, and range of motion categories for a user are weighted differently than one or more of the other categories by the score computing module 240 based on determinations made by the system 10.

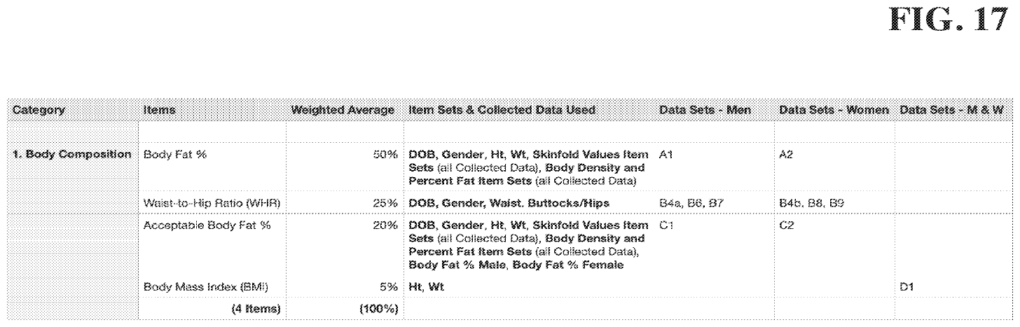

In an embodiment, the total average for the body composition category may be computed based on quantified and weighted averages of the following components: (1) body fat percentage; (2) waist-to-hip ratio; (3) body fat percentage ranges; and (4) body mass index. In a non-limiting example and as best seen in FIG. 17, body fat percentage is weighted as 50%, waist-to-hip ratio is weighted as 25%, acceptable body fat is weighted at 20%, and body mass index is weighted as 5%. In this example, the score computing module 240 gives greater weight to body fat percentage than the other three components based on decisions made by the system 10. In other embodiments, the computing module 240 may generate different percentages for the various components or for other components.

The score computing module 240 is also configured to use the averages and percentages extrapolated from the associated comparative data to provide a new, averaged unique score for one or more of the components of the five main categories. In an example, the comparative data is used to compute a new, averaged score for the body fat percentage, waist-to-hip ratio, body fat percentage ranges, and body mass index components of the body composition category.

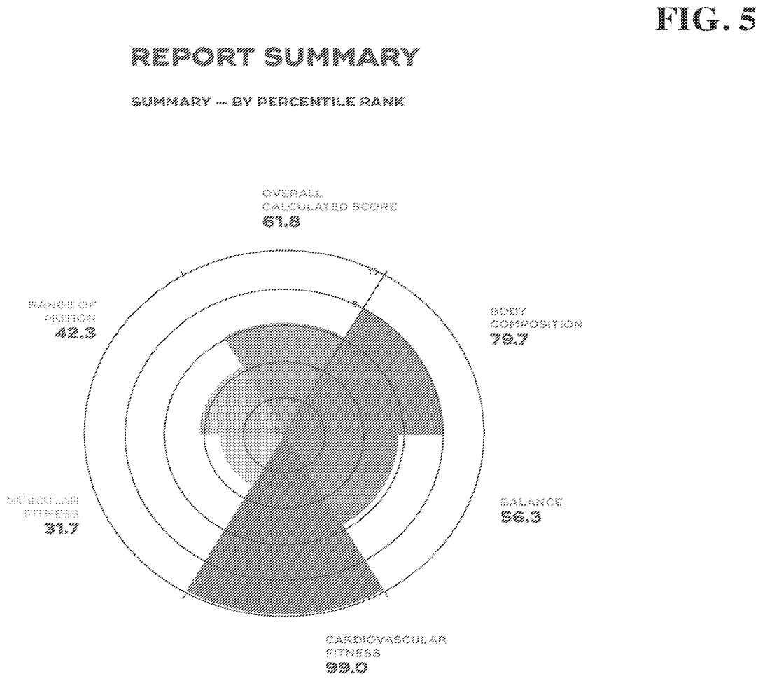

As best seen in FIG. 5, the score computing module 240 is further configured to assign a composite/total score to each user based on the averaged, weighted totals of the five main categories (body composition, balance, cardiovascular/cardiopulmonary fitness, muscular fitness, and range of motion) and all associated comparative data. The composite/total score may be expressed in percentile form and may comprise all tested values, items, components and research. As a result, a user may be able to readily visualize a single percentage number for their overall health and fitness levels. The single percentage number/score is unique for each user.

FIG. 3 illustrates a flow chart illustrating an example method 300 for measuring an individual's health and fitness levels using the system 10. The method commences at block 310 with a user accessing the system 10. In order to obtain access to the system 10, the user creates a user profile using the user account module 210 and provides certain personal data and information, such as their date of birth and gender, to the system 10. The user's data and information is stored on the servers 50 and the databases 95. If a user has already created a user profile with the system 10, the user logs into the system 10 to obtain access.

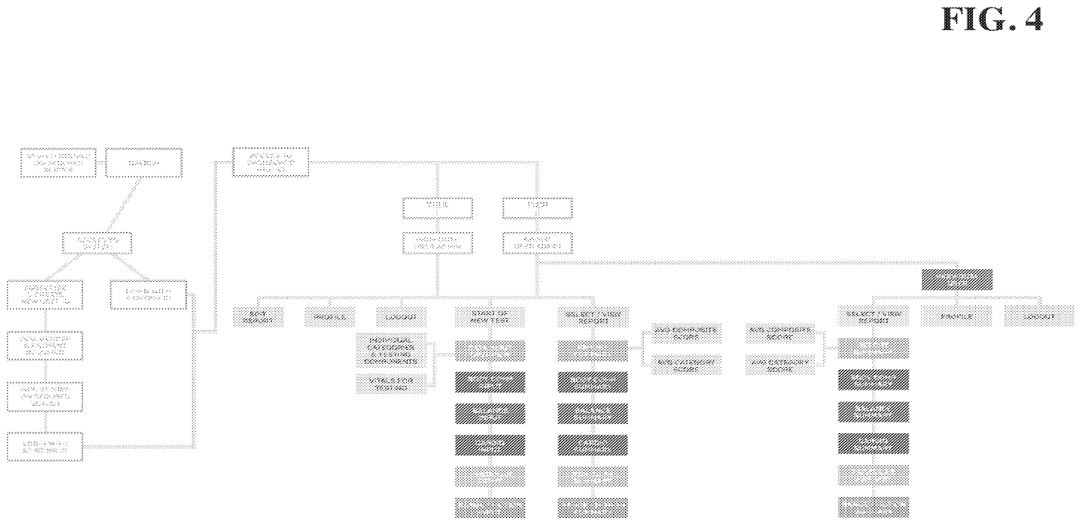

As best seen in FIG. 4 and as described above, the accounts for the system 10 may be an individual user administrator account or a group user administrator account. The group user administrator account includes a plurality of individual users, wherein the group user administrator may view, input, and edit reports regarding the individual users of the group. The individual users under the group user administrator account may only access their user profile information and select/view reports.

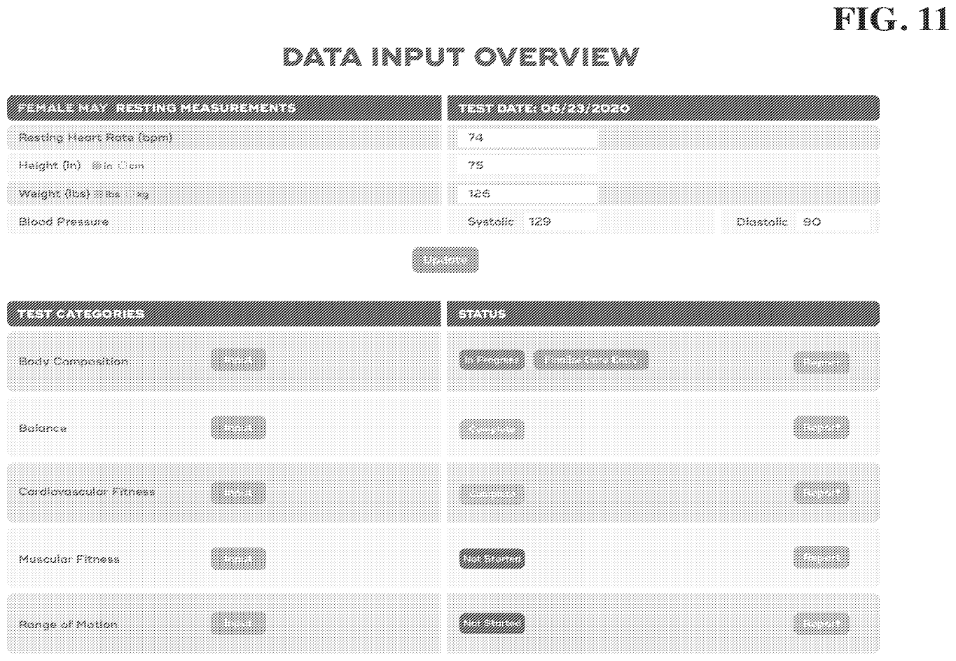

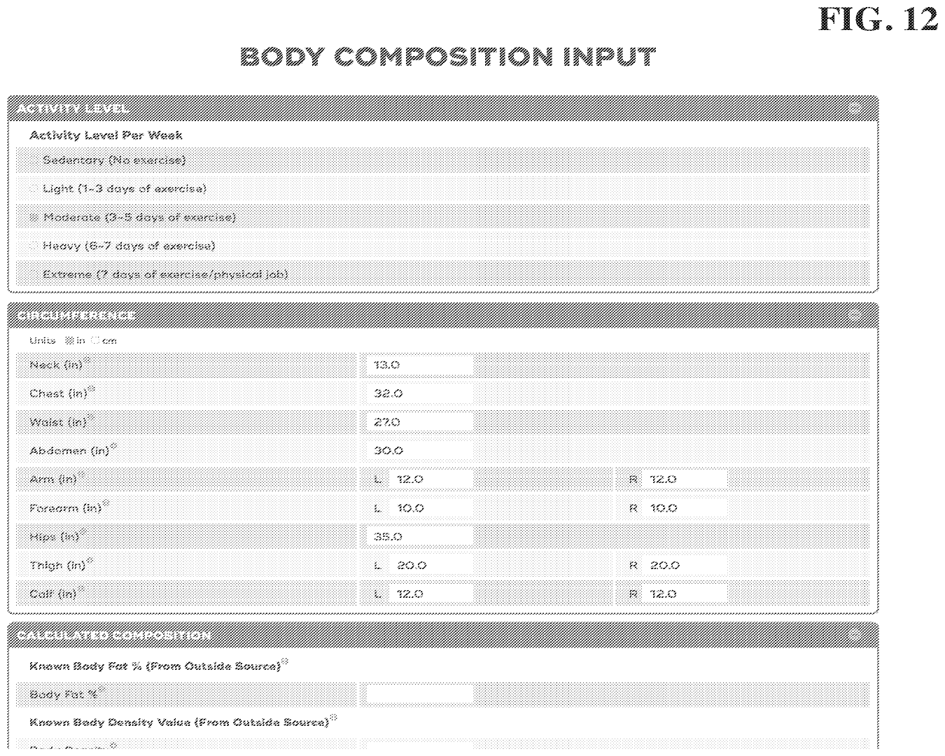

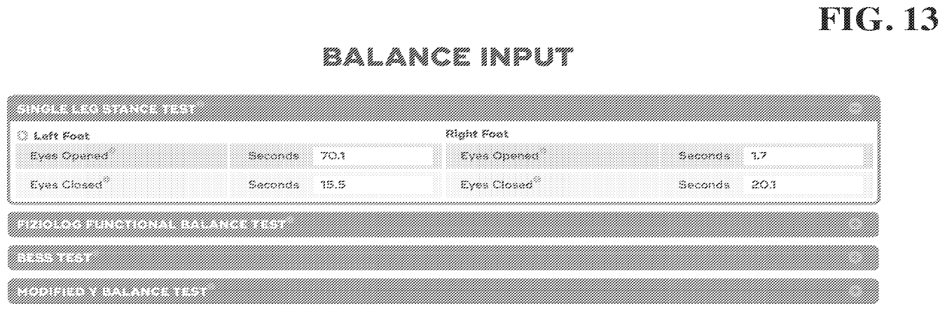

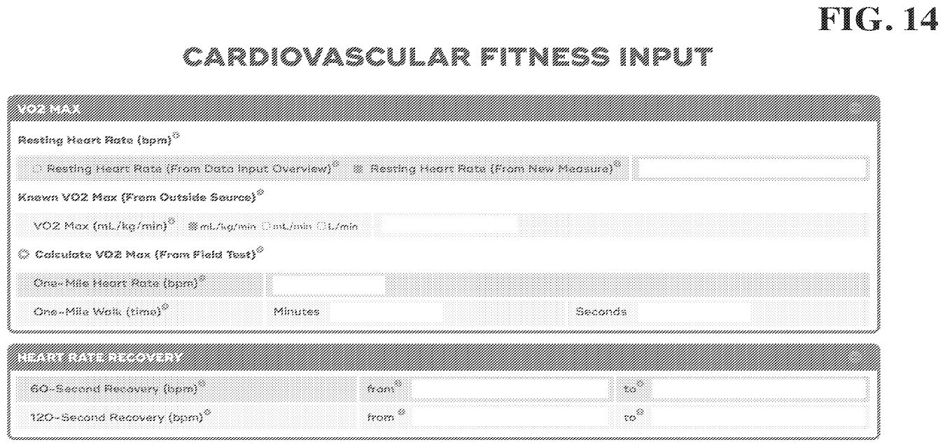

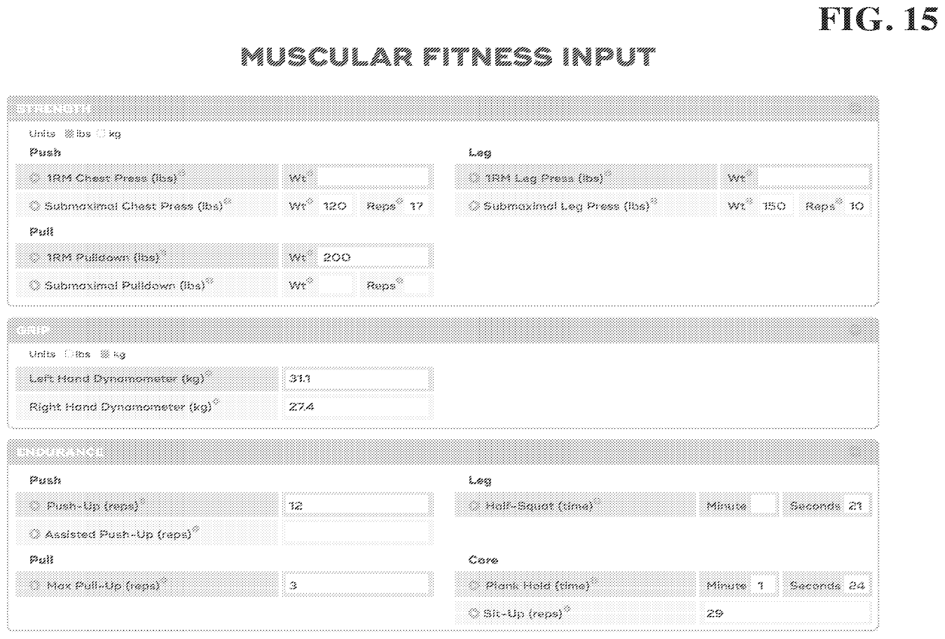

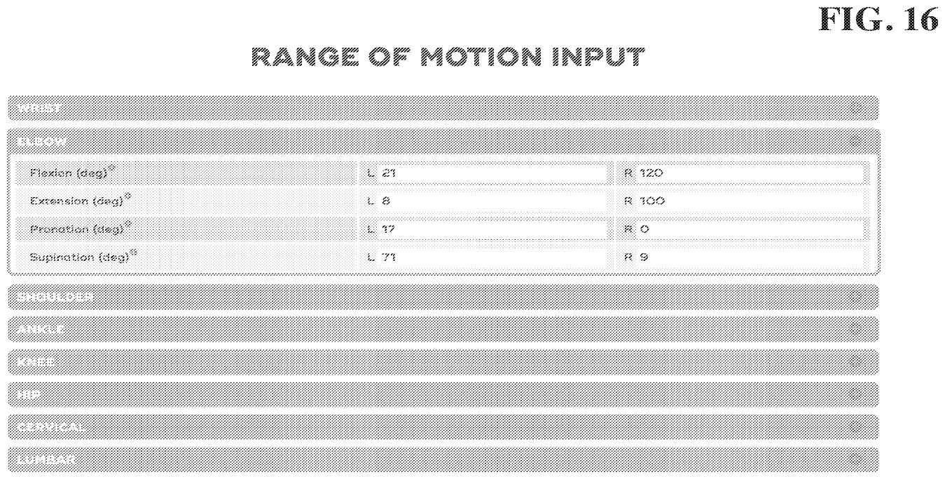

Next, as shown in block 320, the user creates a new session/test in order to input data and/or information into the system 10. The user may readily access or refer to any of their individual sessions/tests. A date/time stamp is generated by the system 10 once the user creates a new session/test. The user may determine the amount of data and/or information they input into the system 10 based on the amount of data and/or information they would like tested or calculated by the health and fitness data analysis module 220. As a result, the user does not need to complete every test or item to generate a category score or an overall/composite score. As the user generates more frequent sessions/tests in the system 10, the user will be able to view multiple averages over significant lengths of time allowing the user to see improvements/regressions, strengths and weaknesses, and overall health and fitness measures. As best seen in FIGS. 11-16, a user may input a variety of different data and information pertaining to their health and fitness using the questions and categories generated by the system 10.

In an alternative embodiment, the second computing device 40 may obtain data and information pertaining to a user's health and fitness from an administrator of a group user account. In other alternative embodiments, data and information regarding a user may be transmitted to the second computing device 40 through software programs, mobile software applications, cameras, sensors, and other devices, such as wearables, webcams, and fitness machines.

Next, as shown in block 330, the health and fitness data analysis module 220 conducts tests and/or calculates scores using the data and information provided by the user. Specifically, the health and fitness data analysis module 220 uses algorithms and equations stored in the servers 50 to generate averages and calculate scores. The health and fitness analysis engine 100 determines which of the five health and fitness categories and which components of the respective categories should be analyzed and how they should be weighted/quantified. This analysis is based on the fields of health and fitness industry standards and trends, published data, and currently recognized criteria that helps identify the level of importance when identifying and measuring health and fitness metrics. For example, a user's body composition may be calculated based on data relating to a user's activity level and the circumferences of one or more of a user's body parts.

Next, as shown in block 340, appropriate comparative data is regularly and continuously identified and incorporated into the databases 95 of the second computing device 40 via the comparative analysis module 230. The comparative data may be selected from one or more scientific publications (e.g., peer-reviewed publications), journal articles, or the like. In an embodiment, each piece of comparative data may be broken down to form a percentile range, such as a percentage from 99-1%. The comparative data may be continually updated, replaced, or evolved as new information, industry standards, and sources become recognized, available, and published.

The comparative data is then compared and contrasted, via the comparative analysis module 230, with the health and fitness data calculated for a user, as shown in block 350. The comparative analysis module 230 uses specific demographic groups to give an accurate representation of where the user stands related to their respective comparative group. The result is a quantifiable classification of a user's overall health and fitness levels based on demographic groups, such as age and gender.

As shown in block 360, a composite health and fitness score is assigned to each user via the score computing module 240. The composite score is determined based on the averaged, weighted totals of each of the five main categories calculated for the user. As best seen in FIG. 5, the composite score may be expressed in percentile form to allow the user to visualize one percentage number for their overall health and fitness levels. In other embodiments, there may be different amounts of categories calculated for the user.

It is to be understood that the various embodiments described in this specification and as illustrated in the attached drawings are simply exemplary embodiments illustrating the inventive concepts as defined in the claims. As a result, it is to be understood that the various embodiments described and illustrated may be combined from the inventive concepts defined in the appended claims.

In accordance with the provisions of the patent statutes, the present disclosure has been described to represent what is considered to represent the preferred embodiments. However, it should be noted that this disclosure can be practiced in other ways than those specifically illustrated and described without departing from the spirit or scope of this disclosure.

1. A system for measuring health and fitness levels of an individual, the system comprising:
a computing device comprising:
a storage medium configured to store data and information associated with health and fitness; and
an engine comprising:
a user account module configured to build user profiles and to authenticate users in the system;
a data analysis module in communication with the user account module, wherein the data analysis module is configured to quantifiably analyze a first set of data and information based on a plurality of health and fitness categories;
a comparative analysis module in communication with the data analysis module, wherein the comparative analysis module is configured to review and evaluate a second set of data and information from one or more external sources, and to compare and contrast the analyzed first set of data and information with the second set of data and information; and
a score computing module in communication with the comparative analysis module, wherein the score computing module is configured to generate a score associated with each of the health and fitness categories based on the input received from the comparative analysis module.

2. The system of claim 1, wherein each of the health and fitness categories comprises a plurality of quantifiably measured components.

3. The system of claim 1, wherein the health and fitness categories comprise body composition, balance, cardiovascular/cardiopulmonary fitness, muscular fitness, and range of motion.

4. The system of claim 1, wherein the second set of data and information and the first set of data and information are each derived from individuals having about the same age and/or gender.

5. The system of claim 4, wherein the second set of data and information is translated into a specific percentile range.

6. The system of claim 2, wherein the score computing module is further configured to compute averages of the components associated with each of the health and fitness categories.

7. The system of claim 6, wherein the score computing module is further configured to:
generate a first score based upon the computed averages of the components associated with the health and fitness categories; and
generate a second score specific for an individual based on the averaged, weighted totals of each of the health and fitness categories.

8. The system of claim 1, wherein the external sources comprise one or more organizations, publications, journals, or academic literature.

9. A method for measuring health and fitness levels of an individual, the method comprising:
configuring an engine having one or more processors to execute instructions for:
creating a new session to receive a first set of data and information pertaining to an individual's health and fitness;
analyzing the first set of data and information to generate averages and to compute scores based on a plurality of health and fitness categories;
identifying and screening a second set of data and information from one or more external sources;
comparing and contrasting the analyzed first set of data and information with the second set of data and information; and
generating a total score for the individual based on computed scores for each of the plurality of health and fitness categories.

10. The method of claim 9, wherein each of the health and fitness categories comprises a plurality of quantifiably measured components.

11. The method of claim 10, wherein the engine is further configured to determine which of the components from the plurality of components and which of the categories from the plurality of health and fitness categories to include in the total score.

12. The method of claim 9, wherein the health and fitness categories comprise body composition, balance, cardiovascular/cardiopulmonary fitness, muscular fitness, and range of motion.

13. The method of claim 9, wherein the external sources comprise one or more organizations, publications, journals, or academic literature.

14. The method of claim 9, wherein the second set of data and information and the first set of data and information are each derived from individuals having about the same age and/or gender.

15. The method of claim 14, wherein the second set of data and information is translated into a specific percentile range.

16. A system for measuring health and fitness levels of an individual, the system comprising:
at least one measurement device configured to capture a first set of health data;
a computing device in communication with the at least one measurement device, the computing device comprising:
a storage medium configured to store data and information associated with health and fitness; and
a processing engine comprising:
a user account module configured to register a plurality user profiles and to authenticate users in the system;
a data analysis module in communication with the user account module, wherein the data analysis module is configured to quantifiably analyze the first set of health data based on a plurality of health and fitness categories;
a comparative analysis module in communication with the data analysis module, wherein the comparative analysis module is configured to review and evaluate a second set of data and information from one or more external sources, and compare and contrast the analyzed first set of data and information with the second set of data and information; and
a score computing module in communication with the comparative analysis module, wherein the score computing module is configured to generate a score associated with each of the health and fitness categories based on the input received from the comparative analysis module.

17. The system of claim 16, wherein the health and fitness categories comprise body composition, balance, cardiovascular/cardiopulmonary fitness, muscular fitness, and range of motion.

18. The system of claim 16, wherein the at least one measurement device is configured to detect physiological attributes of a user associated with one of the user profiles.

19. The system of claim 16, wherein the at least one measurement device comprises a mobile electronic device comprising a software application configured to access and update a first user profile of the plurality of user profiles.

20. The system of claim 16, wherein the at least one measurement device comprises a plurality of health devices, wherein each of the health device detects one a plurality of different health metrics; and
wherein the data analysis module of the processing engine processes the plurality of different health metrics as a second set of health data in combination with the first set of health data based on the plurality of health and fitness categories.
